(19) United States
(12) Patent Application Publication
(10) Pub. No.: US 2026/0199095 A1
(43) Pub. Date: Jul. 16, 2026

(54) BIG DATA-BASED ARTIFICIAL ORGAN PRODUCTION SYSTEM

(71) Applicant: ALDAVER INC., Daejeon (KR)

(72) Inventor: Hyeok Ju CHAE, Daejeon (KR)

(73) Assignee: ALDAVER INC., Daejeon (KR)

(21) Appl. No.: 19/135,203

(22) PCT Filed: Nov. 14, 2023
(86) PCT No.: PCT/KR2023/018235

§ 371 Date: Jun. 3, 2025

(30) Foreign Application Priority Data
Dec. 7, 2022 (KR) 10-2022-0169461

(51) Int. Cl. A61F 2/30; B29C 64/393; B33Y 50/02; B33Y 70/00; B33Y 80/00

(52) U.S. Cl. CPC A61F 2/30942; B29C 64/393; B33Y 50/02; B33Y 70/00; B33Y 80/00; A61F 2002/30943; A61F 2002/30985; A61F 2240/002

(57) ABSTRACT

The present invention relates to a big data-based artificial organ production system. The artificial organ production system according to the present invention includes: an input unit that receives user's requirements for the artificial organ to be produced as input values; a database unit that stores data necessary for producing the artificial organ; a control unit that produces the artificial organ by inputting the input values into the artificial organ production algorithm learned on the basis of the database unit; and a bioprinter that produces the artificial organ under the control of the control unit.

### TECHNICAL FIELD

The present application claims the benefit of priority to Korean Patent Application No. 10-2022-0169461 filed on Dec. 7, 2022, the entire contents of which are incorporated as part of this specification.

The present invention relates to an artificial organ production system, and more particularly, to a system for producing artificial organs on the basis of big data.

### BACKGROUND ART

Bioprinting is being utilized as a method of producing artificial organs. Bioprinting enables the spatial structuring of living cells and other biological products, biomaterials, biochemical substances, or biocompatibility through layer-by-layer deposition under computer control, by sequentially arranging them to develop living tissues and organs for tissue engineering, regenerative medicine, pharmacokinetics, and more generally, biological research.

When attempting to produce artificial organs using such bioprinting, production is carried out after experiment in order to ensure that the artificial organs meet the desired conditions.

Artificial organs are used in various cases, and among the cases, they are also used for medical training. In the case of artificial organs used for training, artificial organs for various cases are needed. However, there is a limitation in producing artificial organs through research and experiment whenever artificial organs suited to various cases are needed.

### PRIOR ART DOCUMENT

### Patent Document

(Patent Document 1) Korean Patent No. 10-1954953 (Registered on Feb. 27, 2019)

### DISCLOSURE

### Technical Problem

Accordingly, an object of the present invention is to provide a big data-based artificial organ production system that can produce artificial organs corresponding to the conditions requested by the user on the basis of big data related to artificial organs.

### Technical Solution

In order to achieve the above object, the present invention provides a big data-based artificial organ production system including: an input unit that receives user's requirements for the artificial organ to be produced as input values; a database unit that stores big data necessary for producing the artificial organ; a control unit that calculates process variables for producing the artificial organ by inputting the input values into the artificial organ production algorithm learned based on the database unit; and a bioprinter configured to produces the artificial organ using the process variables under the control of the control unit.

The input values may include hardness, tensile strength, toughness, and ductility of the artificial organ.

The input values may be numeral values.

The database unit may store data on a plurality of raw materials required for artificial organ production, weight of each raw material, pretreatment conditions, temperature, catalyst conditions, and reaction time.

The database unit may store mechanical property data, which comprises Young's modulus, ultimate tensile strength, toughness, and fracture point, depending on the ratio of a plurality of raw materials when synthesizing the raw materials.

The database unit may store data on agarose, acrylamide, N,N′-methylenebis (acrylamide) (MBA), tetramethylethylenediamine (TEMED), deionized water, ammonium sulfate (APS), and temperature.

The bioprinter may be a 3D bioprinter.

The control unit may create artificial organ modeling files (STL) using the artificial organ production algorithm learned with the input values, and bio-print the artificial organ by providing the artificial organ modeling files (STL) to the bioprinter.

The artificial organ production algorithm may include: a data reverse-engineering model that creates virtual data necessary for machine learning using reverse-engineering technology; and the artificial organ production model that performs machine learning with virtual data created by the data reverse-engineering model, and then, produces a user-customized artificial organ by controlling the bioprinter using user's requirements as input values.

The data reverse-engineering model may include: a surrogate model that extracts features from raw materials through molecular fingerprints and predicts target physical properties (mechanical properties, electrocautery characteristics, and 3D printability) ; a reverse-engineering deep learning model that performs selection of process variables and raw materials that enable synthesis of target physical properties; and a virtual data model that estimates data distribution of a synthesis variable space and creates virtual data.

Further, the artificial organ production model may include: a machine learning model that derives optimal design for each organ tissue on the basis of user's requirements (physiological (BMI, 3D tissue model), genetic, and demographic (age, sex) information of a patient) ; an artificial intelligence model that designs an automated synthesis process including process variables through machine learning on the basis of the big data;
and a printer control model that produces a user-customized artificial organ by applying the process variables to control the bioprinter.

### Advantageous Effects

According to the present invention, it is possible to produce artificial organs corresponding to conditions requested by a user on the basis of big data related to artificial organs. That is, it is possible to produce user-customized artificial organs through bioprinting by using conditions requested by a user as input values on the basis of big data accumulated in the process of producing various artificial organs.

Since the input values are numerical values for the hardness, tensile strength, toughness, and ductility of artificial organs, it is possible to produce artificial organs corresponding to input values in custom-made types by inputting numerical data, and there is an advantage of enabling mass production of artificial organs having the same specifications through a single input.

Since the artificial organ production system according to the present invention is based on big data, it can reduce the time required for experiment, production, and verification.

### DESCRIPTION OF DRAWINGS

FIG. 1 is a block diagram illustrating a big data-based artificial organ production system according to an embodiment of the present invention.

FIG. 2 is a block diagram illustrating the database unit of FIG. 1.

FIG. 3 to FIG. 5 are graphs illustrating the stress-strain characteristics of a monomer (Agarose, Acrylamide) and an additive (MBA).

FIG. 6 is a graph illustrating the mechanical properties of an additive, including Young's modulus, ultimate tensile strength, and toughness.

FIG. 7 is a graph illustrating a fracture point among the mechanical properties of the additive.

FIG. 8 is a table illustrating a data processing result of raw materials used in artificial organ producing.

FIG. 9 is a graph illustrating mechanical properties by ratio of a monomer (Agarose, Acrylamide) and an additive (MBA).

FIG. 10 is a block diagram illustrating an artificial organ production algorithm according to an embodiment of the present invention.

FIG. 11 is a detailed block diagram illustrating a model that constitutes the artificial organ producing algorithm of FIG. 10.

### BEST MODEL

It should be noted that, in the following description, only parts required to understand embodiments of the present invention are described and other parts are omitted without departing from the scope of the present invention unclear.

The terms and words used in the present specification and claims should not be construed as limited to ordinary or dictionary terms, and should be construed in a sense and concept consistent with the technical idea of the present invention, based on the principle that the inventor can properly define the concept of a term to describe his invention in the best way possible. Therefore, since the configurations shown in the embodiments and drawings described in the present specification are only preferred embodiments of the present invention, and does not represent all of the technical spirit of the present invention, it should be understood that various equivalents and modifications may be substituted for them at the time of filing the present application.

Hereinafter, embodiments of the present invention are described in more detail with reference to the accompanying drawings.

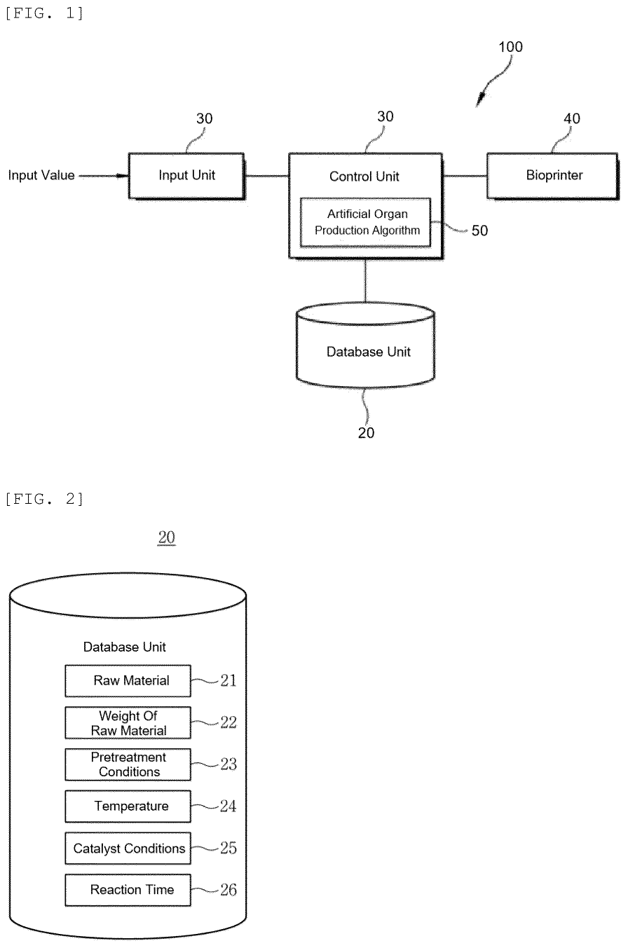

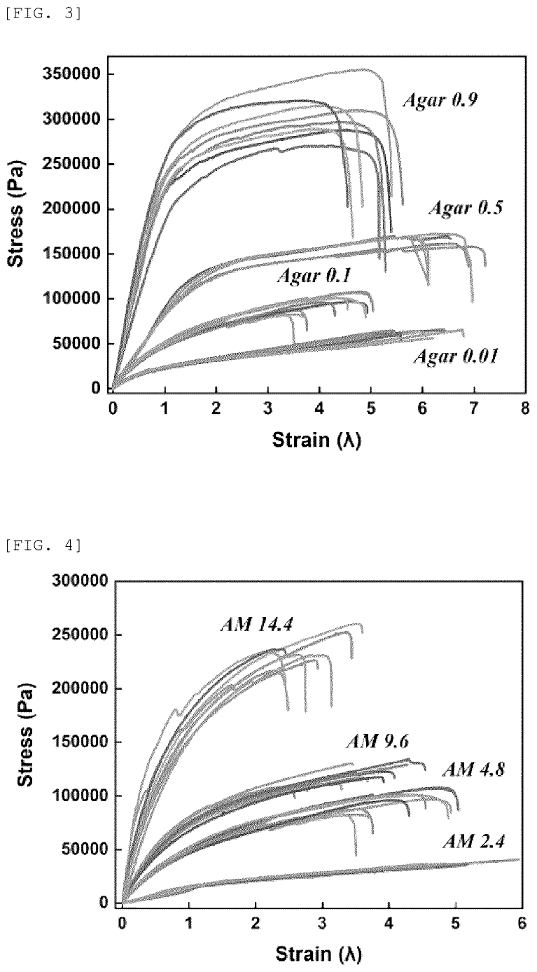

FIG. 1 is a block diagram illustrating a big data-based artificial organ production system according to an embodiment of the present invention. Further, FIG. 2 is a block diagram illustrating the database unit of FIG. 1.

Referring to FIG. 1 and FIG. 2, an artificial organ production system 100 according to this embodiment is a system that produces user-customized artificial organs through bioprinting using an artificial organ production algorithm 50 learned on the basis of big data.

The artificial organ production system 100 according to this embodiment includes an input unit 10, a database unit 20, a control unit 30, and a bioprinter 40. The input unit 10 receives user's requirements for an artificial organ to be produced as input values. The database unit 20 stores big data necessary for producing artificial organs. The control unit 30 derives process variables that enable production of artificial organs by inputting input values into the artificial organ production algorithm 50 learned on the basis of the database unit 20. Further, the bioprinter 40 produces an artificial organ using process variables under the control of the control unit

In this configuration, the input unit 10 receives requirements for an artificial organ to be produced as input values from a user. The user's requirements may include patient's physiological (BMI, three-dimensional tissue model), genetic, and demographic (age, sex) information. The input values may include information on the type and physical properties of artificial organs. The information on physical properties includes the hardness, tensile strength, toughness, and ductility of artificial organs, and corresponding input values are input as numerical values.

Alternatively, the information on physical properties may be input in textual form. The control unit 30 can convert the information on physical properties input in textual form into numerical values on the basis of big data.

The input unit 10 may be a physical input device, such as a keyboard or mouse, that directly receives requirements from a user. Alternatively, the input unit 10 may be a communication unit that receives requirements through a user terminal.

The database unit 20 stores big data used for production of various artificial organs. This database unit 20 stores data on a plurality of raw materials necessary for artificial organ production, the weight of each raw material, pretreatment conditions, temperature, catalyst conditions, and reaction time. For example, the database unit 20 may store data necessary for artificial organ production, such as agarose, acrylamide, N,N′-methylenebis (acrylamide) (MBA), tetramethylethylenediamine (TEMED), deionized water, ammonium sulfate (APS), and temperature, which are materials according to an embodiment, and the present invention is not limited thereto.

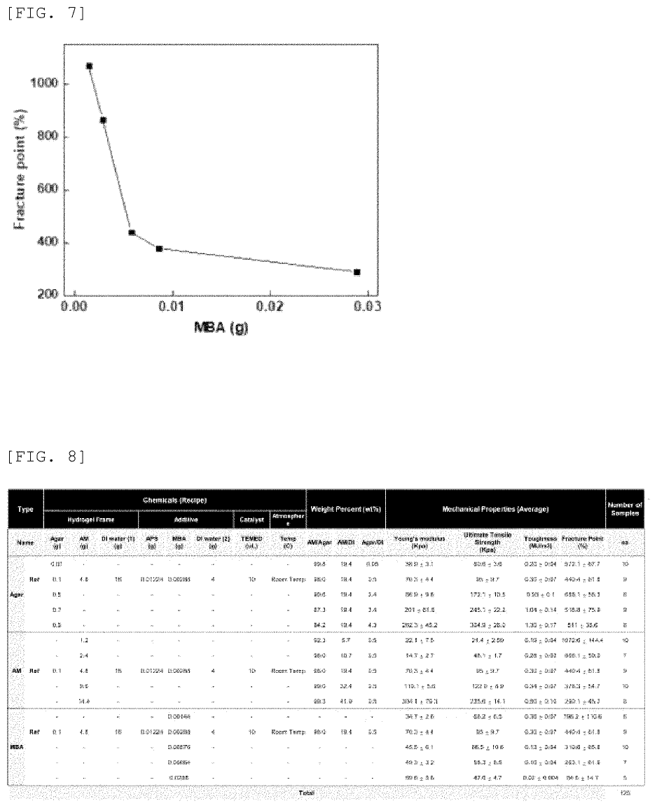

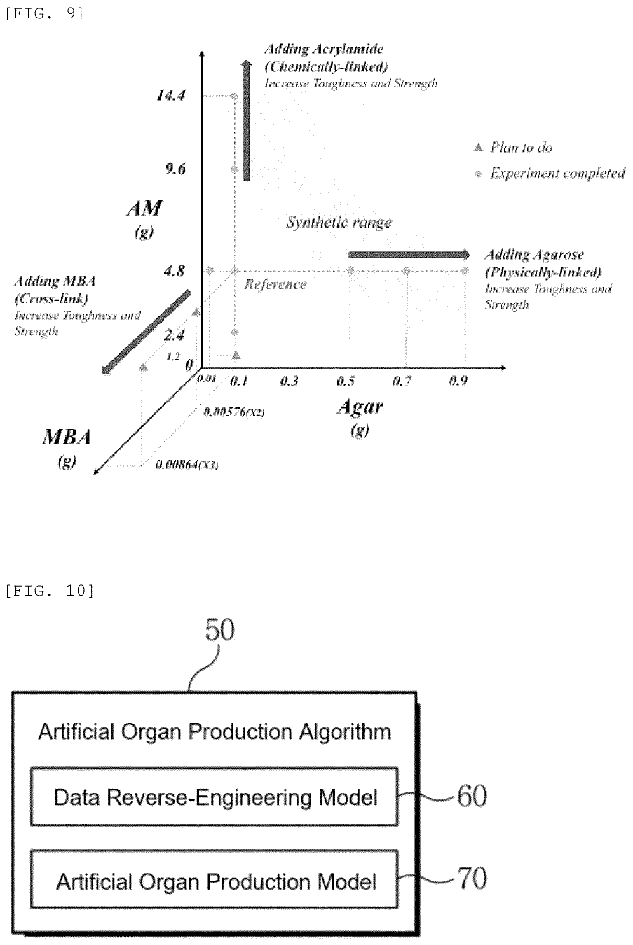

As big data, as shown in FIG. 3 to FIG. 9, when synthesizing raw materials that are used in the production of artificial organs, mechanical property data (Young's modulus, ultimate tensile strength, toughness, fracture point) is collected on the basis of the ratio of monomers (Agarose, Acrylamide) or additives (MBA). In FIG. 8, ‘Agar’ represents Agarose and ‘AM’ represents Acrylamide.

It can be observed that as the amount of monomers and additives increases when synthesizing raw materials, toughness and strength increase.

Accordingly, in order to produce artificial organs, synthesis conditions (process variables) of the raw materials suitable for the user's requirements can be derived through machine learning on the basis of the mechanical property data according to the amount of the above-mentioned monomers and additives when synthesizing raw materials.

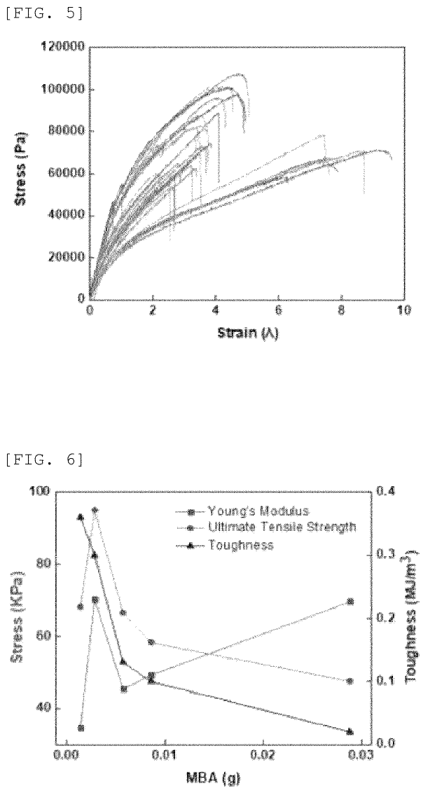

FIG. 3 to FIG. 5 are graphs illustrating the stress-strain characteristics of a monomer (Agarose, Acrylamide) and an additive (MBA) ·FIG. 3 illustrates the stress-strain characteristic of Agarose. FIG. 4 illustrates the stress-strain characteristic of Acrylamide. Further, FIG. 5 illustrates the stress-strain characteristic of an additive (MBA).

The database unit 20 can store artificial organ modeling files (STL) created using the artificial organ production algorithm 50 that has been learned with user's input requirements as input values.

The bioprinter 40 produces artificial organs on the basis of the artificial organ production algorithm 50 that has been learned under the control of the control unit 30. The bioprinter 40 is a 3D bioprinter. That is, the bioprinter 40 receives artificial organ modeling files (STL) created by the learned artificial organ production algorithm 50 and produces artificial organs through 3D bioprinting.

In the present invention, artificial organs are produced through 3D bioprinting and may include, for example, artificial skin, artificial blood vessels (for bleeding and hemostasis), and artificial peritoneum, but are not limited to these.

3D bioprinting is a process of obtaining an image by scanning a target structure in three dimensions, and then creating a scanned image into a three-dimensional structure using cells and bioink. The typical 3D printing technologies include extrusion-based processes, inkjet-based processes, and laser-based processes, among which the extrusion-based process is primarily used in bioprinting to create three-dimensional structures with cells. Since bioprinting involves living cells, bioink, which is a substance that forms a three-dimensional structure by mixing with cells, is the most important part of 3D bioprinting.

On the other hand, when artificial organs according to this embodiment are utilized for medical education, not all of the raw materials constituting bioink need to be biocompatible substances.

Further, the control unit 30 is a microprocessor that performs overall control of the artificial organ production system 100. The control unit 30 controls the production of user-CUSTOMIZED artificial organs through bioprinting using the artificial organ production algorithm 50 learned on the basis of big data. That is, the control unit 30 creates an artificial organ modeling files (STL) using the artificial organ production algorithm 50 learned with user's requirements for artificial organs to be produced as input values. Further, the control unit 30 provides artificial organ modeling files (STL) to the bioprinter 40, thereby producing user-customized artificial organs through bioprinting.

The artificial organ production algorithm 50 according to this embodiment uses a method of analyzing trends on the basis of big data and then reverse-engineering raw materials by selecting an algorithm with high prediction rate. Further, the artificial organ production algorithm can provide user-customized artificial organs using machine learning to be able to minimize trial and error depending on various user requirements, such as sex and age.

In this case, the machine learning may include various algorithms, and appropriate algorithms can be selected depending on data. Machine learning algorithms include supervised learning, unsupervised learning, semi-supervised learning, and reinforcement learning.

Supervised learning involves learning from data that includes labels in the data that a computer learns. In this case, the label refers to a correct answer value for data, and the computer determines which answer value is similar to a new input. That is, supervised learning is a method of learning from data using data that has correct answers.

Algorithms for such supervised learning include k-Nearest Neighbors, Linear Regression, Logistic Regression, Gaussian Process Regression, Support Vector Machine (SVM), Decision Tree, Random Forest, Neural Network, and so on, but are not limited thereto.

Unsupervised learning involves learning from data that does not include labels in the data that a computer learns. That is, unsupervised learning is a method in which input data does not contain correct answers and thus a computer predicts a result for new data by clustering (grouping) similar items.

Algorithms for such unsupervised learning include clustering, visualization, dimensionality reduction, and association rule learning, but are not limited thereto. Clustering may include k-means, Hierarchical Cluster Analysis (HCA), and expectation maximization. Visualization and dimensionality reduction may include Principal Component Analysis (PCA), kernel PCA, Locally-Linear Embedding (LLE), t-distributed Stochastic Neighbor Embedding (t-SNE), etc. Further, association rule learning may include Apriori, Eclat, etc.

Semi-supervised learning is a combination of supervised learning and unsupervised learning and performs leaning from data some of which include labels. That is, semi-supervised learning is a learning method that uses labels only for some data when it is intended to improve the performance of supervised learning but there is a limitation in resources for labeling data.

Further, reinforcement learning performs learning through trial and error by taking an action in a given environment and receiving feedback from the action. Reinforcement learning performs learning by allowing a program to make decisions without providing explicit data to a machine learning model, and by receiving feedback on whether the decisions were correct or incorrect. In this case, the feedback is called a reward, and learning is performed in such a way that decisions are made to receive positive rewards in subsequent decisions and to avoid making decisions that lead to negative rewards.

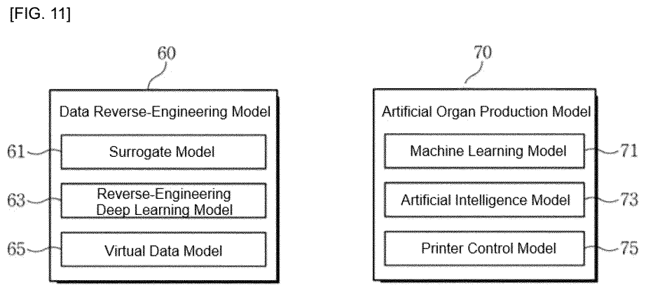

This artificial organ fabrication algorithm 50 includes a data reverse-engineering model 60 and an artificial organ production model 70, as shown in FIG. 10 and FIG. 11. FIG. 10 is a block diagram illustrating the artificial organ production algorithm 50 according to an embodiment of the present invention. FIG. 11 is a detailed block diagram illustrating a model that constitutes the artificial organ producing algorithm 50 of FIG. 10.

The data reverse-engineering model 60 creates data necessary for learning the artificial organ production model 70 using reverse-engineering technology.

Further, the artificial organ production model 70 performs machine learning with the data created by the data reverse-engineering model 60, and then, produces a user-customized artificial organ by controlling the bioprinter 40 using user's requirements as input values.

The data reverse-engineering model 60 is described as follows.

In general, since the biological properties of human organs are very different for each individual patient, it is necessary to secure raw materials with various physical properties to produce artificial organs in order to secure a large number of customers. However, due to the diversity and complexity of the properties of raw materials, it takes significant efforts, both in terms of time and cost, to directly synthesize all the raw materials with target physical properties. Accordingly, in this embodiment, the reverse-engineering technology of raw materials through machine learning was used on the basis of the data reverse-engineering model 60.

The data reverse-engineering model 60 created virtual data as follows to train the artificial organ production model 70. That is, in order to address the lack of experimental data and improve the performance of reverse-engineering models, the data reverse-engineering model 60 employed a technology that creates new data points (virtual data) on-demand.

This data reverse-engineering model 60 may include a surrogate model 61, a reverse-engineering deep learning model 63, and a virtual data model 65. In this case, the data reverse-engineering model 60 was developed with the goal of achieving a target physical property prediction accuracy of 90% and a reverse-engineering process variable estimation accuracy of 80%.

The surrogate model 61 extracts features from raw materials through molecular fingerprints and predicts the target physical properties (mechanical properties, electrocautery characteristics, 3D printability).

The reverse-engineering deep learning model 63 performs selection of process variables and raw materials that enables synthesis of target physical properties.

Further, the virtual data model 65 estimates data distribution of a wide synthesis variable space and creates virtual data.

The artificial organ production model 70 includes a machine learning model 71, an artificial intelligence model 73, and a printer control model 75.

Optimal design parameters of artificial organs based on the user (patient)'s physiological conditions, age, sex, etc. are very different, but a method of predicting these parameters and providing custom-made artificial organs has not existed in the past. Even if optimal target design parameters are determined, experimental data that is used by the data reverse-engineering model 60 for machine learning has limitations in estimating all of the spaces of all variables.

Accordingly, in this embodiment, the artificial organ production model 70 establishes an efficient process of estimating design parameters of target organs based on the conditions of various patients and systematically modifying the producing process and material composition ratios from the limited data.

The artificial organ production model 70 was produced with the goal of achieving 80% accuracy in predicting patient's optimized design variables, and an average of 5 iterations for the modification of production processes and compositions to obtain target variables.

The machine learning model 71 derives optimal design for each organ tissue on the basis of user's requirements, such as various physiological (BMI, 3D tissue model, etc.), genetic, and demographic (age, sex, etc.) information of various patients.

The artificial intelligence model 73 can design an automated synthesis process including process variables through various types of machine learning on the basis of big data. The artificial intelligence model 73 can select and use an appropriate machine learning algorithm in accordance with the data included in big data. For example, the artificial intelligence model 73 can design an automated synthesis process that includes optimized process variables on the basis of Gaussian processes and Bayesian methods, but is not limited thereto.

Further, the printer control model 75 produces user-customized artificial organs by applying the process variables designed by the artificial intelligence model 73 to control of the 3D bioprinter.

The process of producing artificial organs by inputting input values into the artificial organ production algorithm 50 as described above is described as follows using an example.

Further, artificial organs are produced through 3D bioprinting by inputting artificial organ modeling files (STL) into the 3D bioprinter.

According to this embodiment, it is possible to produce artificial organs corresponding to conditions requested by a user on the basis of big data related to artificial organs. That is, it is possible to produce user-customized artificial organs through bioprinting by using a user's requirements as input values on the basis of big data accumulated in the process of producing various artificial organs.

Since the input values are numerical values for the hardness, tensile strength, toughness, and ductility of artificial organs, it is possible to produce artificial organs corresponding to input values by inputting numerical data, and this provides an advantage of enabling mass production.

Further, since the artificial organ production system 100 according to this embodiment is based on big data, it can reduce the time required for experiment, production, and verification.

Meanwhile, this specification and the embodiments illustrated in the drawings are merely to provide specific examples for helping understanding of the present invention, without limiting the scope of the present invention. It is apparent to those skilled in the art that the present invention may be modified in various ways on the basis of the technical spirit of the present invention other than the embodiments disclosed herein.

1. A big data-based artificial organ production system, comprising:
an input unit that receives user's requirements for the artificial organ to be produced as input values;
a database unit that stores big data necessary for producing the artificial organ;
a control unit that calculates process variables for producing the artificial organ by inputting the input values into the artificial organ production algorithm learned based on the database unit; and
a bioprinter configured to produces the artificial organ using the process variables under the control of the control unit.

2. The big data-based artificial organ production system according to claim 1, wherein the input values include hardness, tensile strength, toughness, and ductility of the artificial organ.

3. The big data-based artificial organ production system according to claim 1, wherein the input values are numeral values.

4. The big data-based artificial organ production system according to claim 1, wherein the database unit stores data on a plurality of raw materials for artificial organ production, weight of each raw material, pretreatment conditions, temperature, catalyst conditions, and reaction time.

5. The big data-based artificial organ production system according to claim 4, wherein the database unit stores mechanical property data, which comprises Young's modulus, ultimate tensile strength, toughness, and fracture point, depending on the ratio of a plurality of raw materials when synthesizing the raw materials.

6. The big data-based artificial organ production system according to claim 1, wherein the database unit stores data on agarose, acrylamide, N,N′-methylenebis (acrylamide) (MBA), tetramethylethylenediamine (TEMED), deionized water, ammonium sulfate (APS), and temperature.

7. The big data-based artificial organ production system according to claim 1, wherein the bioprinter comprises a 3D bioprinter.

8. The big data-based artificial organ production system according to claim 1, wherein the control unit creates artificial organ modeling files (STL) including the process variables using the artificial organ production algorithm learned with the input values, and bio-prints the artificial organ by providing the artificial organ modeling files (STL) created to the bioprinter.

9. The big data-based artificial organ production system according to claim 1, wherein the artificial organ production algorithm includes:
a data reverse-engineering model that creates virtual data necessary for machine learning using reverse-engineering technology; and
the artificial organ production model that performs machine learning with virtual data created by the data reverse-engineering model, and then, produces a user-customized artificial organ by controlling the bioprinter using user's requirements as input values.

10. The big data-based artificial organ production system according to claim 9, wherein the data reverse-engineering model includes:
a surrogate model that extracts features from raw materials through molecular fingerprints and predicts target physical properties (mechanical properties, electrocautery characteristics, and 3D printability) ;
a reverse-engineering deep learning model that performs selection of process variables and raw materials that enable synthesis of target physical properties; and
a virtual data model that estimates data distribution of a synthesis variable space and creates virtual data.

11. The big data-based artificial organ production system of claim 10, wherein the artificial organ production model includes:
a machine learning model that derives optimal design for each organ tissue on the basis of user's requirements (physiological (BMI, 3D tissue model), genetic, and demographic (age, sex) information of a patient) ;
an artificial intelligence model that designs an automated synthesis process including process variables through machine learning on the basis of the big data; and
a printer control model that produces a user-customized artificial organ by applying the process variables to control of the bioprinter.
